(19) United States
(12) Patent Application Publication
(10) Pub. No.: US 2023/0372574 A1
(43) Pub. Date: Nov. 23, 2023

(54) IN VIVO DETECTION OF SURGICAL MATERIALS

(71) Applicant: Northwestern University, Evanston, IL (US)

(72) Inventors: Thomas J. Meade, Evanston, IL (US); Vinayak P. Dravid, Evanston, IL (US)

(21) Appl. No.: 18/200,932

(22) Filed: May 23, 2023

Related U.S. Application Data
(60) Provisional application No. 63/344,694, filed on May 23, 2022.

(51) Int. Cl. A61L 15/54; A61L 15/18

(52) U.S. Cl. CPC A61L 15/54; A61L 15/18; A61L 2400/12; A61L 2420/02

(57) ABSTRACT

Provided herein are surgical materials detectable by multiple imaging modalities, including magnetic resonance imaging (MRI) and computerized tomography (CT). In some embodiments, provided herein are surgical materials comprising bismuth nanoparticles and/or iron oxide nanoparticles.

### STATEMENT REGARDING RELATED APPLICATIONS

This application claims priority to U.S. Provisional Patent Application No. 63/344,694, filed May 23, 2022, the entire contents of which are incorporated herein by reference for all purposes.

### FIELD

Provided herein are surgical materials detectable by multiple in-vivo imaging modalities, including magnetic resonance imaging (MRI) and computerized tomography (CT). In some embodiments, provided herein are surgical materials comprising bismuth nanoparticles and/or iron oxide nanoparticles.

### BACKGROUND

Estimates for surgeries worldwide are over 300 million per year, near 100 million in the United States and Europe. Foreign items left inside patients is an unfortunate scenario estimated to occur in between 0.1 and 0.3% of surgeries, with consequences that may not appear until months or years have passed. This rate is likely underreported for fear of legal ramifications. Among the most common surgical items left behind in patients are surgical sponges and gauze, as they become difficult to visually distinguish after use, and their small size and flexible nature allow for displacement especially in the abdominal cavity. Complications from lost gauze and surgical sponges can include fistula, hernia, and wound sepsis, resulting in additional surgeries and recovery time for patients, and malpractice suits against doctors reducing access to operating rooms and increasing the cost of healthcare for prospective patients.

Current solutions to reducing the cases of foreign objects left in patients after surgeries include counting all objects used and removed during the surgery, including an electronic tag on surgical sponges and gauze used in surgery, and including a radiopaque tracer in the surgical sponges and gauze used in surgery that can be detected by intraoperative X-Ray imaging. Each of these detection methods comes with its own challenges and downsides. The radiopaque tracers present in a large percentage of surgical sponges and gauzes have remained unchanged for the past 80 years. While the BaSO₄ polymer present in these radiopaque tracers provides X-ray contrast, it can become separated from the surgical gauze and post-operative imaging is increasingly moving away from X-ray imaging in favor of MRI. The electronic tags that have been included in studies to investigate detection of retained surgical sponges are both expensive and bulky, while suffering from the same attachment concerns as current radiopaque tracers. The counting method for preventing retained surgical items has seen the greatest adoption; however, despite the best efforts of surgical staff, counts of used and recovered items are frequently incorrect.

MR imaging is used in the majority (60-70%) of post-operative follow-up surveillance in the United States. However, there is currently a lack of surgical gauze that can be detected by MRI. U.S. Patent Publication No. 2021/0259894 describes a surgical gauze with a planar material and a detectable element detectable by MRI, but does not describe gauzes detectable by MRI or CT. U.S. Pat. No. 10,905,521 describes a surgical article with a radioopaque material and absorbant material with an elongated cut ribbon partially radiolucent and partially radiopaque, but similarly does not disclose materials for MRI or CT detectable gauzes.

Existing surgical materials that are only detectable by CT scan expose the subject to unnecessary radiation. Moreover, clinically used CT contrast agents such as iodinated organic compounds, barium sulfate suspensions for GI imaging, and the commercially available barium sulfate contrast strip used in radiopaque surgical gauze are inefficient and the molecular agents require high doses to deliver sufficient CT contrast. Accordingly, what is needed are inexpensive and safe surgical materials that can be efficiently detected by MRI and CT.

### SUMMARY

In some aspects, provided herein are surgical materials. In some embodiments, provided herein are surgical materials comprising a detectable coating detectable by magnetic resonance imaging (MRI) and/or computerized tomography (CT). In some embodiments, the detectable coating comprises bismuth nanoparticles and/or iron oxide nanoparticles. In some embodiments, provided herein are surgical materials comprising a detectable coating detectable by magnetic resonance imaging (MRI) and computerized tomography (CT). In some embodiments, the detectable coating comprises bismuth nanoparticles and iron oxide nanoparticles.

In some embodiments, the detectable coating comprises Fe₃O₄ nanoparticles. In some embodiments, the detectable coating comprises bismuth nanoparticles and Fe₃O₄ nanoparticles. In some embodiments, the surgical material comprises a fibrous material. The surgical materials described herein may be used in a subject, including a human subject.

In some aspects, provided herein is a composition comprising a plurality of bismuth nanoparticles. In some embodiments, the composition further comprises a plurality of iron oxide nanoparticles. The composition finds use in methods of making a surgical material. In some embodiments, provided herein is method of making a surgical material comprising a detectable coating, the method comprising contacting the surgical material with a composition comprising a plurality of bismuth nanoparticles. In some embodiments, the composition further comprises a plurality of iron oxide nanoparticles.

In some aspects, provided herein is a method of producing a bismuth nanoparticle, the method comprising dissolving a bismuth precursor and a surfactant in a composition comprising at least one alcohol, and reducing the bismuth precursor with a suitable reducing agent. In some embodiments, the bismuth precursor comprises Bi(NO₃)₃ and the surfactant comprises polyvinylpyrrolidone. In some embodiments, the composition comprising at least one alcohol comprises glycerol and ethanol. In some embodiments, the reducing agent comprises NaBH₄.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows cellulose threads coated with BiNPs and backed by Styrofoam. FIG. 1B shows X-ray CT imaging of the bismuth NP coated cellulose gauze threads compared to a commercially available barium containing CT contrast strip (far right). Note that the commercially available barium containing strip used for comparative purposes in the figures and examples herein is significantly thicker than the treated gauze fibers developed and tested herein. ICP analysis of representative samples from 1, 2, and 3 (threads shown from left to right in the figure) measured 125, 180, and 180 μg Bi/mg coated gauze respectively. The CT images contrast is very surprising considering they are single threads.

FIG. 2 (top panel) shows cellulose gauze threads coated with Fe₃O₄ nanoparticles and suspended in agarose. FIG. 2 (bottom panel) shows MR imaging of the Fe₃O₄ coated gauze threads. ICP analysis of representative samples from 1, 2, 3, (threads shown from left to right) and C (far right) measured 2, 10, 15, and 0 μg Fe/mg coated thread respectively.

FIG. 3A and FIG. 3B show schematic diagrams showing exemplary methods by which gauze coated with bismuth nanoparticles and iron oxide nanoparticles (e.g., Bi/Fe₃O₄ NPs) may be prepared.

FIG. 4 shows representative images comparing the resulting gauze prepared by the various procedures highlighted in FIG. 3.

FIG. 5A shows an exemplary CT image of Bi/Fe₃O₄ NP coated gauze fibers, embedded in 2% agarose. FIG. 5B shows an exemplary MR image of Bi/Fe₃O₄ NP coated gauze fibers, embedded in 2% agarose. Fibers treated with 240 to 360 μg Bi/mg gauze (e.g. 30% bismuth, 36% bismuth, 24% bismuth, and 29% bismuth) demonstrated clearly visible X-ray CT contrast. Samples 1-3 coated with 9 and 3 μg iron/mg treated gauze demonstrate visible MR contrast, while sample 4 with only 1.5 μg Fe/mg treated gauze is difficult to distinguish even in the relatively uniform background provided by agarose.

FIGS. 6A-6C are exemplary images demonstrating in-vivo efficacy Bi/Fe₃O₄ NP coated gauze fibers placed in an exemplary animal tissue (e.g. chicken thigh). FIG. 6A shows an exemplary CT image following placement of coated fibers within the tissue. FIG. 6B shows placement and alignment of the coated fibers within the tissue under natural lighting. FIG. 6C shows an exemplary MR image (T₂ weighted MR) of the coated fibers following placement within the tissue.

FIGS. 7A-7C are exemplary images demonstrating in-vivo efficacy of Bi/Fe₃O₄ NP coated gauze fibers placed in a different exemplary animal tissue (e.g. bovine tissue, namely steak containing connective tissue). Fibers are aligned as shown above in FIG. 6. FIG. 7A shows an exemplary CT image of the coated fibers following placement within the tissue. FIG. 7B shows placement and alignment of the fibers within the tissue under natural lighting. FIG. 7C show an exemplary MR image (T₂ weighted MR) of the coated fibers following placement within the tissue.

FIG. 8 show exemplary images of Bi/Fe₃O₄ NP coated gauze fibers. Shown are T₂ weighted MR imaging alone (right) and with CT overlaid (left) in bovine tissue with no connective tissue (e.g. tenderloin). Gauze threads were placed singly and twisted together to mimic potential imaging scenarios.

FIG. 9 show exemplary T₂ weighted MR images of Fe₃O₄ treated gauze fibers. Fibers contained varying amounts of Fe₃O₄, as identified above the respective images.

FIG. 10 is a graph showing loading of barium salts (BaCl₂) and bismuth salts (BiCl3) on gauze fibers. Fibers were treated as identified in the figure and the amount of respective salts (in ng/mg fiber) was determined.

FIG. 11A shows an exemplary CT image of gauze fibers coated with barium salts (BaCl₂) or bismuth salts (BiCl₃). FIG. 11B shows an exemplary CT image demonstrating CT contrast for a commercially available, thick polymer fiber coated with barium.

FIG. 12A and FIG. 12B demonstrate exemplary loading conditions tested for creating BiCl₃ coated gauze fibers.

FIGS. 13A-13D show exemplary conditions tested for creating BiCl3 and MNS coated fibers. FIG. 13A is a schematic showing one exemplary method tested herein, wherein gauze was incubated with MNS and BiCl₃ (simultaneously) for one hour, followed by rolling the samples. Following rolling, samples were allowed to dry, then dialyzed for 12 hours in water, then dried again to create the final product. FIG. 13B is a schematic showing another exemplary method tested herein, wherein gauze was incubated first with BiCl₃ for 1 hour, then allowed to dry, followed by incubating with MNS for 1 hour. After incubation with MNS, gauze was dried, dialyzed for 12 hours in water, then dried again create the final product. FIG. 13C is a schematic showing another exemplary method tested herein. Gauze was drop cast with Fe₃O₄ MNS, allowed to dry, then drop cast with BiCl₃. Gauze was then dried, dialyzed for 24 hours in water, and dried again to create the final product. FIG. 13D is a schematic showing another exemplary method tested herein. Gauze was drop cast with Fe₃O₄ MNS and BiCl₃ (simultaneously) allowed to dry, dialyzed for 24 hours in water, and dried again to create the final product.

### DETAILED DESCRIPTION

### 1. Definitions

Although any methods and materials similar or equivalent to those described herein can be used in the practice or testing of embodiments described herein, some preferred methods, compositions, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the terms “comprise”, “include”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject. In some embodiments, the subject is undergoing or has undergone surgery.

### 2. Surgical Materials and Methods of Use Thereof

In some aspects, provided herein are surgical materials. The term “surgical material” as used herein refers to any article that comes into contact with a subject during a surgical procedure. For example, a “surgical material” may refer to an implement such as a knife, a scalpel, a blade, a surgical staple, a needle, etc. As another example, a “surgical material” may refer to a fibrous material, including gauze, dressings, sponges, surgical thread (e.g., sutures) bandages, wrappings, grafts, stents, catheters, etc. In some embodiments, the fibrous material is a woven material.

In some embodiments, provided herein are surgical materials comprising a detectable material which may be a coating. As used herein, the term “coating” refers to a composition that at least partially encapsulates or adheres to the surgical material. For example, for a fibrous material, the detectable coating encapsulates or adheres to at least a portion of the fibers, such that a portion of the external surface of the fibers is covered with the detectable coating.

In some embodiments, the detectable coating is detectable by magnetic resonance imaging (MRI). For example, in some embodiments the detectable coating is detectable using an MRI device. As used herein, the term “magnetic resonance imaging (MRI) device” or “MRI” incorporates all devices capable of magnetic resonance imaging or equivalents. The methods of the invention can be practiced using any such device, or variation of a magnetic resonance imaging (MRI) device or equivalent, or in conjunction with any known MRI methodology. For example, in magnetic resonance methods and apparatuses, a static magnetic field is applied to a tissue or a body under investigation in order to define an equilibrium axis of magnetic alignment in a region of interest. A radio frequency field is then applied to that region in a direction orthogonal to the static magnetic field direction in order to excite magnetic resonance in the region. Magnetic field gradients are applied to spatially encode the signals. The resulting signals are detected by radio-frequency coils placed adjacent to the tissue or area of the body of interest. See, e.g., U.S. Pat. Nos. 6,144,202; 6,128,522; 6,127,775; 6,119,032; 6,111,410; 5,555,251; 5,455,512; 5,450,010, each of which is herein incorporated by reference in its entirety. MRI and supporting devices are manufactured by, e.g., Bruker Medical GMBH; Caprius; Esoate Biomedica; Fonar; GE Medical Systems (GEMS); Hitachi Medical Systems America; Intermagnetics General Corporation; Lunar Corporation; MagneVu; Marconi Medicals; Philips Medical Systems; Shimadzu; Siemens; Toshiba America Medical Systems; and Varian: including imaging systems, by, e.g., Silicon Graphics. Magnetic Resonance Imaging (MRI) is a non-invasive imaging technology that produces three dimensional detailed anatomical images. MRI is a preferred imaging technique due to its tunable soft-tissue contrast, high spatial and temporal resolution, and lack of ionizing radiation.

In some embodiments, the detectable coating is detectable by X-ray imaging. The term “x-ray” or “x-ray imaging” is used in the broadest sense and refers to any imaging based on the absorption of x-rays. In some embodiments, the detectable coating is detectable by computerized tomography (CT). CT is an imaging methodology used to create detailed images of internal organs, bones, soft tissue, and blood vessels. CT is a computerized x-ray imaging procedure that combines a series of x-ray images taken from different angles and uses computer processing to create cross-sectional images. Accordingly, a detectable coating detectable by CT scan is also considered to be detectable by x-ray imaging. Cross-sectional images generated during a CT scan can be formatted in multiple planes and used to generate three-dimensional images.

In some embodiments, provided herein are surgical materials comprising a detectable coating, wherein the detectable coating is detectable by MRI and/or CT. Accordingly, the surgical materials provided herein are advantageous over non-coated surgical materials currently used in the art, which are only detectable by CT and therefore require exposing the subject, perhaps unnecessarily, to radiation.

In some embodiments, the detectable coating comprises a contrast agent. As used herein, the term “contrast agent” refers to any agent which enhances visibility of one or more structures in a medical imaging procedure. In some embodiments, the detectable coating comprises an MRI contrast agent. In some embodiments, the detectable coating comprises a metal ion. For example, in some embodiments the detectable coating comprises a paramagnetic metal ion. As used herein, the terms “paramagnetic metal ion”, “paramagnetic ion” or “metal ion” refer to a metal ion that is magnetized parallel or antiparallel to a magnetic field to an extent proportional to the field. Generally, these are metal ions that have unpaired electrons. Examples of suitable paramagnetic metal ions, include, but are not limited to, gadolinium III (Gd+3 or Gd(III)), iron III (Fe+3 or Fe(III)). manganese II (Mnt2 or Mn(II)), yttrium III (Yt+3 or Yt(III)), dysprosium (Dy+3 or Dy(III)), and chromium (Cr(III) or Cr+3). In some embodiments, the metal ion comprises a superparamagnetic metal ion. As used herein, the term “superparamagnetic” refers to a form of magnetism which appears in sufficiently small particles. Superparamagnetic materials have a larger magnetic susceptibility than paramagnetic materials. In some embodiments, superparamagnetic materials comprise iron, such as iron oxide or iron platinum particles. In some embodiments, the contrast agent comprises iron oxide nanoparticles (NPs). In some embodiments, the iron oxide nanoparticles comprise Fe₃O₄ NPs or Fe₂O₃ NPs. In some embodiments, the iron oxide nanoparticles are doped. For example, in sonic embodiments the iron oxide nanoparticles are doped (e.g. alloyed) with magnesium (Mn) and/or zinc (Zn). We For example, in some embodiments the iron oxide nanoparticles comprise ZnMn ferric NPs defined by the formula ZnₓMn₍₁₋ₓ₎Fe₂O₄. In some embodiments, x=0.1-0.4.

In some embodiments, the metal ion is selected from the group consisting of Mn(II), Gd(III), Dy(III), Ho(III), Er(III), Eu(III), Tb(III), Sm(III), Ce(III), Pr(III), Yb(III), Tm(III), Nd(III), and Tb(IV). In some embodiments, the metal ion is Gd(III).

In some embodiments, the metal ion is chelated by a chelation moiety selected from the group consisting of ethylenediaminetetraacetic acid (EDTA), diethylenetriaminepentaacetic acid (DTPA), 1,4,8,11-tetraazacyclododecane-1,4,8,11-tetraacetic acid (TETA), triethylene tetraamine hexaacetic acid (TTHA), 1,4,7,10-tetraazacyclododecane-1,4,7,10-tetraacetic acid (DOTA). TAGA, 1,4,7,10-tetraazacyclododecane-1.4,7,10-tetra(methylene phosphonic acid) (DOTP), diethylenetriaminepentaacetic acid (DTPA), 3,9-bis(methylcarbamoylmethyl)-3,6,9-triazaundecanedioic acid; N,N′-(Carboxymethyliminobisethylene)bis [N-(methylcarbamoylmethyl)glycine]; N,N′-Bis [(methylcarbamoyl)methyl][N,N′-bis(carboxymethyl)[2,2′-(carboxymethylimino)bis(ethaneamine)]] (DTPA-BMA), 1,4,7,10-tetraazacyclkdodecane-1,7-bis(acetic acid) (DO2P), and 1,4,7-tris(carboxymethyl)-1,4,7,10-tetraazacyclododecane (DO3A). In some embodiments, the chelation moiety comprises DO3A.

In some embodiments, the chelation moiety comprises DTPA.

In some embodiments, the detectable coating comprises an MRI contrast agent. Exemplary MRI contrast agents include, but are not limited to. Gd-DOTA. Gd-HP-DO3A, Gd-BT-DO3A, Gd-DTPA, Gd-DTPA-BMEA, Gd-DTPA-BMA, Gd-BORTA, Gd-EOB-DTPA, MS-325, Gd-BOPTA, Mn-DPDP, and Dy-DTPA-BMA.

In some embodiments, the detectable coating comprises a plurality of nanoparticles. The term “nanoparticle” and “nanostructure” are used synonymously to refer to particles having a diameter in all dimensions of between 1 to 100 nanometers. In some embodiments, a “nanoparticle” refers to a particle (e.g. a bismuth particle, an iron oxide particle) having an average diameter of between 1 to 100 nanometers. A magnetic nanoparticle is also referred to interchangeably herein by the abbreviations MNS (magnetic nanostructure), and MNP (magnetic nanoparticle).

In some embodiments, the detectable material comprises one or a plurality of small particles of paramagnetic or superparamagnetic compounds or metals. The particles may be in the form of beads and are generally in the size range of about 1 nanometer to about 100 nanometers in diameter. In some embodiments, the particles have an average diameter of about 1 nanometer to about 100 nanometers. In some embodiments, the particles have an average diameter of about 1 nanometer to about 50 nanometers. In some embodiments, the particles have an average diameter of about 1 nanometer to about 40 nanometers. In some embodiments, the particles have an average size of about 1 nanometer to about 30 nanometers. In some embodiments, the particles have an average size of about 1 nanometer to about 25 nanometers. In some embodiments, the particles have an average size of about 5 nanometers to about 25 nanometers. In some embodiments, the particles have an average size of about 5 nanometers to about 20 nanometers. In some embodiments, the particles have an average size of about 5 nanometers to about 15 nanometers. In some embodiments, the particles have an average size of about 5 nanometers to about 10 nanometers. In some embodiments, the particles have an average size of about 1 nanometer, about 2 nanometers, about 3 nanometers, about 4 nanometers, about 5 nanometers, about 6 nanometers, about 7 nanometers, about 8 nanometers, about 9 nanometers, about 10 nanometers, about 11 nanometers, about 12 nanometers, about 13 nanometers, about 14 nanometers, about 15 nanometers, about 16 nanometers, about 17 nanometers, about 18 nanometers, about 19 nanometers, about 20 nanometers, about 21 nanometers, about 22 nanometers, about 23 nanometers, about 24 nanometers, or about 25 nanometers. The particles can be in the shape of a sphere or a rod. Such particles are encompassed in the meaning of the term nanoparticles as used herein. In some embodiments, the nanoparricles are spherical or substantially spherical. Nanostructures may also be cylindrical, star-shaped, or any other useful shape. All embodiments herein indicated as nanoparticles may also encompass nanostructures (e.g., nanostars), or may be limited to spherical nanoparticles, unless otherwise indicated.

In some embodiments, the detectable coating further comprises one or more polymers. For example, the detectable coating may additionally comprise a biocompatible polymer layer which stabilizes the nanoparticles. The term “nanoparticle” or “NP” as used herein is inclusive of nanoparticles stabilized by the addition of such polymers (e.g. polymer-modified nanoparticles). Exemplary polymers include, but are not limited to, dextran, polyvinyl alcohol, polyethylene oxide, polyethylene glycols, poly-N-isopropylacrylaminde, polyvinylpyrrolidone, polyoligoethylene oxide, polyimine, polyacrylic acid, poly(N,N-dimethylethylamino acrylate), and the like.

In some embodiments, the detectable coating comprises superparamagnetic nanoparticles. In some embodiments, the detectable coating comprises nanoparticles comprising transition metals, including iron, copper, nickel, or alloys thereof. In some embodiments, the detectable coating comprises iron nanoparticles (e.g. Fe3 or Fe(III) nanoparticles, Fe2 or Fe(II) nanoparticles). For example, in some embodiments the detectable coating comprises iron oxide nanoparticles. In some embodiments the detectable coating comprises Fe₃O₄ nanoparticles. In some embodiments the detectable coating comprises Fe₂O₃ nanoparticles. In some embodiments, the detectable coating comprises iron platinum nanoparticles. In some embodiments, the detectable coating comprises MnFe₂O₄ nanoparticles, CoFe₂O₄ nanoparticles, or NiFe₂O₄ nanoparticles. In some embodiments, the iron oxide nanoparticles are doped. For example, in some embodiments the iron oxide nanoparticles are doped (e.g. alloyed) with magnesium (Mn) and/or zinc (Zn). We For example, in some embodiments the iron oxide nanoparticles comprise ZnMn ferric NPs defined by the formula ZnₓMn₍₁₋ₓ₎Fe₂O₄. In some embodiments, x=0.1-0.4.

In some embodiments, the detectable coating comprises iron oxide nanoparticles. In some embodiments, the iron oxide (e.g. Fe₃O₄. Fe2O₃) nanoparticles are prepared by coprecipitation. For example, in some embodiments iron oxide nanoparticles are prepared by providing a suitable iron precursor, such as FeCl₃·6H₂O and/or FeCl₂·4H₂O, and precipitating with a suitable precipitating agent. In some embodiments, the precipitating agent is NaOH.

In some embodiments, the MR can be used for T1 or/and T2 mode of MR imaging. For example, nanoparticles that are smaller (e.g. ˜<4-5 nm) can act as T1 agents while nanoparticles having a larger size (10-15 nm) offer T2 contrast. In some embodiments, the gauze is coated with multiple sizes of iron oxide nanoparticles to offer a contrast agent detectable by T1 and T2 MRI (e.g. dual contrast). For example, in some embodiments the gauze is coated with iron oxide particles having a size of 5 nm or less and coated with iron oxide particles having a size of about 10-15 nm, thus providing a gauze detectable by both T1 and T2 weighted MR imaging.

In some embodiments, the detectable coating comprises a CT contrast agent. In some embodiments, the detectable coating comprises bismuth, iodine, or gold. For example, in some embodiments the detectable coating comprises bismuth nanoparticles, iodine nanoparticles, gold nanoparticles, or polymers comprising the same. In some embodiments, the detectable coating comprises bismuth nanoparticles.

In some embodiments, the nanoparticles (e.g. iron oxide nanoparticles, bismuth nanoparticles) are treated with a capping agent/surfactant. The term “capping agent” and “surfactant” are used interchangeably herein to refer to a stabilizer that inhibits the over-growth of nanoparticles and/or prevents their aggregation/agglomeration. Suitable capping agents include, for example, polyethylene glycol (PEG), polyvinyl alcohol (PVA), silica, bovine serum albumin (BSA), dextran, ethylene diamine tetra acetic acid (EDTA), chitosan, and the like.

In some embodiments, the detectable coating comprises bismuth nanoparticles that are synthesized by chemical reduction using a suitable bismuth precursor and a suitable reducing agent. In some embodiments, nanoparticles are synthesized additionally using a capping agent/surfactant. In some embodiments, the bismuth precursor is Bi(NO₃)₃. In some embodiment, the detectable coating comprises bismuth nanoparticles, and no or substantially no iron oxide(s). In some embodiments, the reducing agent is NaBH₄. In some embodiments, polyvinylpyrrolidone (PVP) is the capping agent/surfactant. Other suitable capping agents are described above.

In some embodiments, the detectable coating comprises an MRI contrast agent and a CT contrast agent. For example, in some embodiments the detectable coating comprises iron oxide nanoparticles and bismuth nanoparticles. For example, in some embodiments the detectable coating comprises Fe₃O₄ nanoparticles and bismuth nanoparticles.

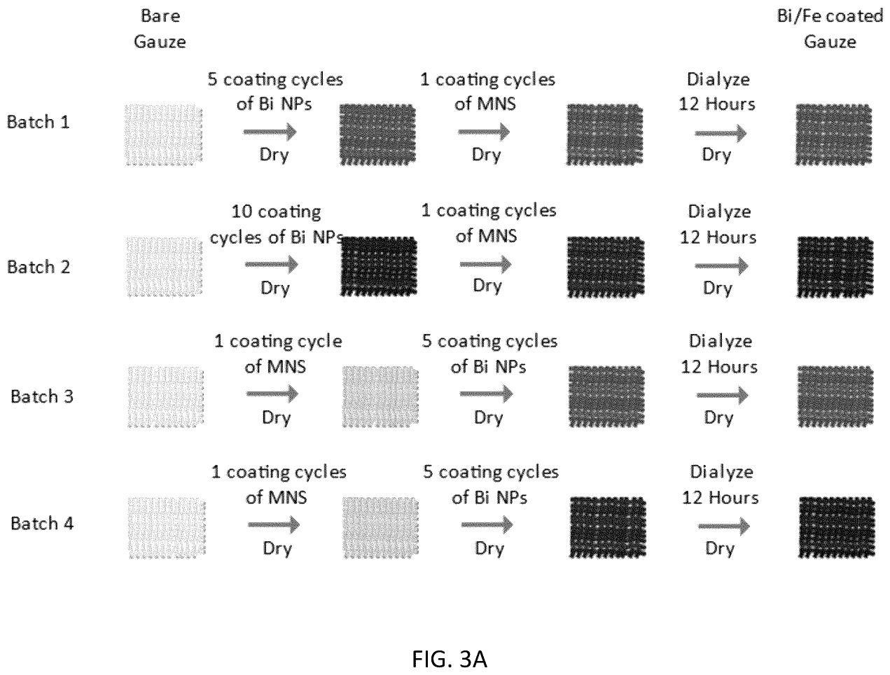

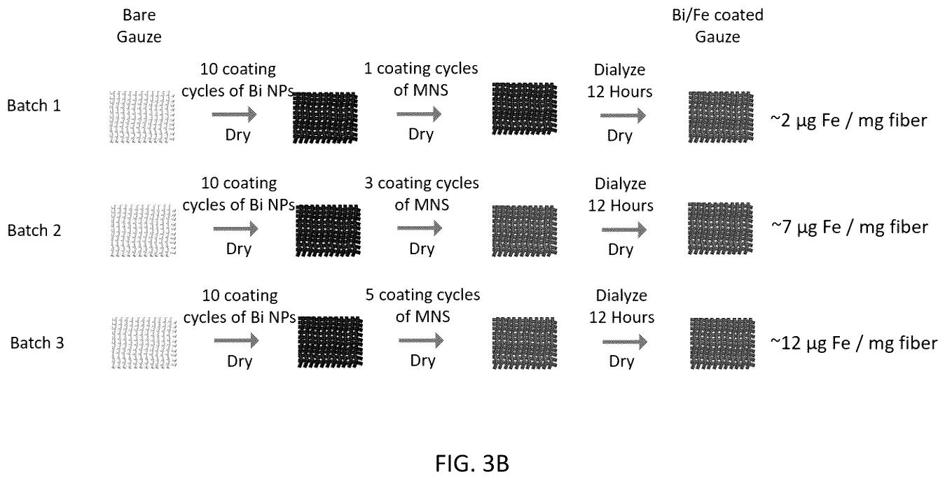

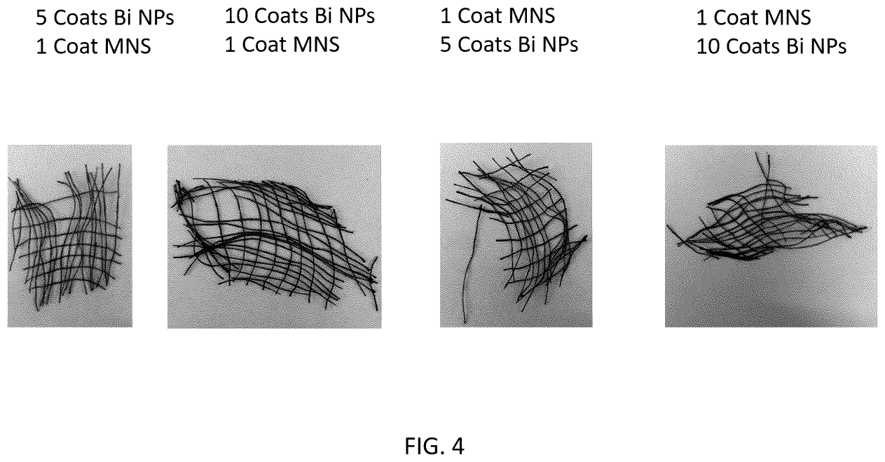

In one embodiment, the detectable coating should comprise a sufficient amount of the contrast agent(s) to facilitate detection of the coating (and therefore facilitate detection of the surgical material comprising the detectable coating) by the desired imaging modality. In some embodiments, the detectable coating comprises a single layer of the contrast agent(s). In some embodiments, the detectable coating comprises multiple layers of the contrast agent(s). The term “layer” as used herein refers to a film of material coated upon the surgical material in a single coating cycle, as described, for example below and as shown in FIG. 3. In some embodiments, the detectable coating comprises 1 to 10 layers of bismuth nanoparticles. For example, in some embodiments the detectable coating comprises 1 layer, 2 layers, 3 layers, 4 layers, 5 layers, 6 layers, 7 layers, 8 layers, 9 layers, or 10 layers of bismuth nanoparticles. In some embodiments, the detectable coating comprises more than 10 layers of bismuth nanoparticles. In some embodiments, the detectable coating comprises one or more layers of bismuth nanoparticles and one or more layers of iron oxide nanoparticles. In some embodiments, the detectable coating comprises 1 to 5 layers of iron oxide nanoparticles. In some embodiments, the detectable coating comprises 1 layer, 2 layers, 3 layers, 4 layers, or 5 layers of iron oxide particles. In some embodiments, the layers of bismuth nanoparticles are separate from the layers of iron oxide nanoparticles. In other words, in some embodiments the surgical material is coated with the bismuth nanoparticles and independently coated with the iron oxide nanoparticles. In some embodiments, the surgical material is coated with one or more layers of contrast agents, wherein each layer comprises both bismuth nanoparticles and iron oxide nanoparticles. In other words, in some embodiments the surgical material is coated with a combined solution containing both bismuth nanoparticles and iron oxide nanoparticles.

For example, suitable procedures for preparing a surgical material as described herein are shown in FIG. 3. In the figure, “MNS” refers to “magnetic nanostructures”, which is another suitable term to describe the magnetic nanoparticles (“MNPs”), described herein, including paramagnetic nanoparticles and superparamagnetic nanoparticles, including polymer-stabilized nanoparticles. For example, the term “MNS” may refer to the iron oxide nanoparticles or polymers described herein.

In some embodiments, the surgical material is prepared performing one or more coating cycles with the nanoparticles described herein. Suitable methods for preparing the surgical material are shown, for example, in FIG. 3. For example, in some embodiments the surgical material is prepared by 1-10 coating cycles with bismuth nanoparticles, and 1-10 coating cycles with iron oxide nanoparticles. In some embodiments, the bismuth nanoparticles and/or iron oxide nanoparticles are suspended in a suitable liquid medium. Suitable liquid mediums include, for example, ethanol and water. In some embodiments, the bismuth nanoparticles are suspended in ethanol. In some embodiments, the iron oxide nanoparticles are suspended in water. The term “coating cycle” refers to the process of submerging the surgical material in the suspension containing the desired nanoparticles. For example, a “coating cycle” with bismuth nanoparticles refers to the process of submerging the surgical material in the suspension containing the bismuth nanoparticles. In some embodiments, excess liquid is removed from the surgical material prior to a subsequent submersion of the surgical material in the suspension. For example, excess liquid may be removed by compressing the surgical material to release excess liquid (e.g. squeezing, wringing, etc.).

In some embodiments, the surgical material is prepared by first contacting the surgical material with the bismuth nanoparticles, and subsequently contacting the surgical material with iron oxide nanoparticles (or other magnetic nanostructures). For example, in some embodiments the surgical material is prepared by performing 1, 2, 3, 4, 5, 6, 7, 8, 9, 10, or more than 10 coating cycles with bismuth nanoparticles. Excess liquid may be removed from the surgical material by compressing the surgical material prior to performing a subsequent coating cycle. In some embodiments, the surgical material is then contacted with iron oxide nanoparticles. For example, after performing a suitable number of coating cycles with a suspension containing bismuth nanoparticles, a suitable number of coating cycles with a suspension containing iron oxide nanoparticles may then be performed.

In other embodiments, the surgical material is prepared by first contacting the surgical material with the iron oxide nanoparticles (or other magnetic nanostructures), and subsequently contacting the surgical material with the bismuth nanoparticles. For example, in some embodiments the surgical material is prepared by performing 1, 2, 3, 4, 5, 6, 7, 8, 9, 10, or more than 10 coating cycles with iron oxide nanoparticles (or other magnetic nanostructures). Excess liquid may be removed from the surgical material by compressing the surgical material prior to performing a subsequent coating cycle. In some embodiments, the surgical material is then contacted with bismuth nanoparticles. For example, after performing a suitable number of coating cycles with a suspension containing iron oxide nanoparticles, a suitable number of coating cycles with a suspension containing bismuth nanoparticles may then be performed.

In some embodiments, the surgical material is dialyzed following performing the desired number of coating cycles. For example, in some embodiments the surgical material is dialyzed for 12-24 hours. In some embodiments, the surgical material is then allowed to dry.

In some embodiments, the surgical material comprises, in total, at least 1 μg of iron per mg of the surgical material. In some embodiments, the surgical material comprises, in total, at least 2 μg of iron per mg of the surgical material. In some embodiments, the surgical material comprises about 2 μg to about 15 μg iron per mg of the surgical material. In some embodiments, the surgical material comprises about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, or about 15 μg iron per mg of the surgical material.

In some embodiments, the surgical material comprises at least about 20% bismuth. In some embodiments, the surgical material comprises about 20% to about 40% bismuth. In some embodiments, the surgical material comprises about 24% to about 36% bismuth. In some embodiments, the surgical material comprises about 24%, about 25%, about 26%, about 27%, about 28%, about 29%, about 30%, about 31%, about 32%, about 33%, about 34%, about 35%, or about 36% bismuth. In some embodiments, the surgical material comprises, in total, at least 1 μg of iron (e.g., Fe(II), Fe(III), per mg of the surgical material and at least about 20% bismuth. In some embodiments, the surgical material comprises about 1 μg of iron to about 10 μg of iron per mg of the surgical material and at least about 20% bismuth (e.g. about 20% to about 40% bismuth).

In some embodiments, the surgical materials described herein are used in a subject, such as during surgery. In some embodiments, the subject is a mammal. In some embodiments, the subject is a human. Use of the surgical materials described herein in a subject during surgery facilitates detection of the materials, such as by MRI and/or CT scan, in the event that they are accidentally left within the subject during the surgical procedure. Accordingly, the surgical materials described herein promote the safety of the subject during surgery, and represent a significant advantage over currently used surgical materials that can only be detected by CT scan, thereby exposing the subject to potentially unnecessary doses of radiation.

### EXAMPLES

### Example 1

Gauze samples were treated with barium salts (BaCl₂) or bismuth salts (BiCl₃) and MNPs to generate coated fibers. Varying concentrations of salts and MNPs were used. Barium coated samples were treated with 1 mg/mL BaCl₂ and 0.28 mg/mL MNP, 5 mg/mL BaCl₂ and 1.4 mg/mL MNP, or 10 mg/mL BaCl₂ and 2.8 mg/mL MNP. Bismith coated samples were treated with varying concentrations of salts and MNPs. Samples were rolled to promote even coating. After coating, samples were washed overnight in ultrapure water and dried.

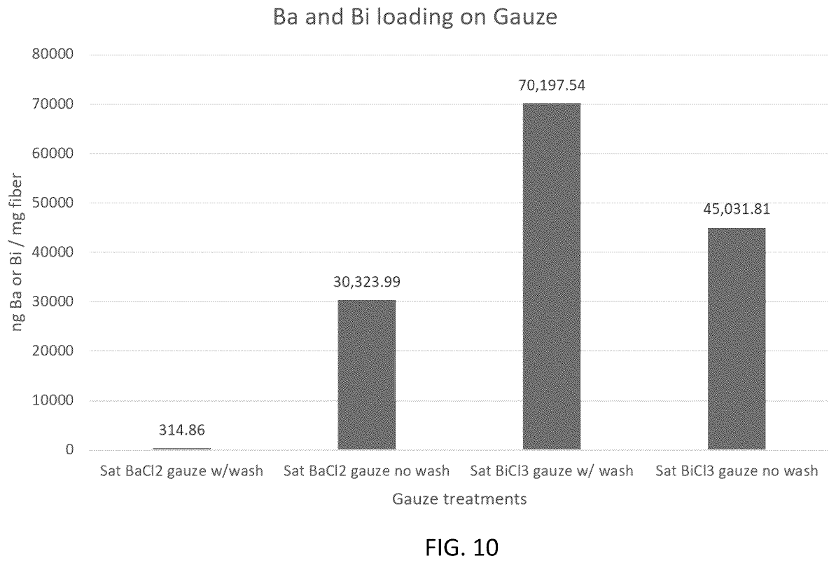

Next, barium (Ba) and bismith (Bi) loading on gauze was evaluated. Results are shown in FIG. 10. As shown in the figure, barium did not bind to the fibers of the gauze stably. In contrast, bismuth was found to stably bind to the gauze.

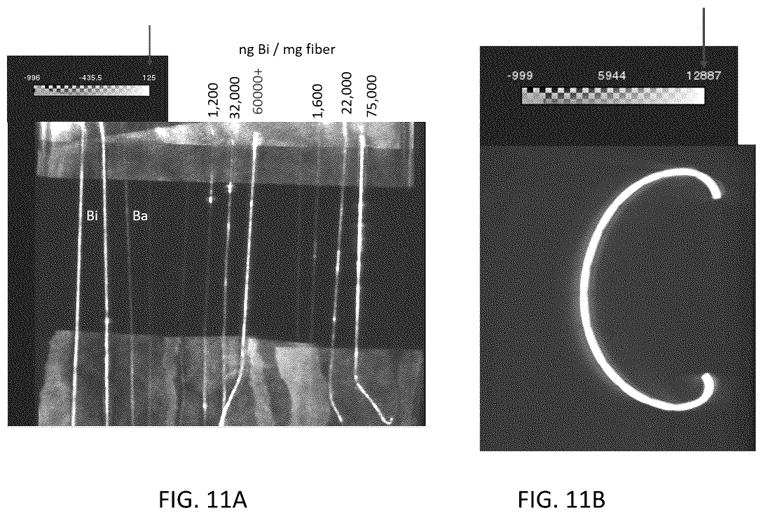

The contrast of barium and bismuth loaded gauze fibers was next measured. Results are shown in FIG. 11. As shown in FIG. 11A, CT contrast of barium treated fibers was barely visible. CT contrast of bismuth treated fibers was visible, but significantly less than the contrast of commercially available barium treated fibers, shown in FIG. 11B.

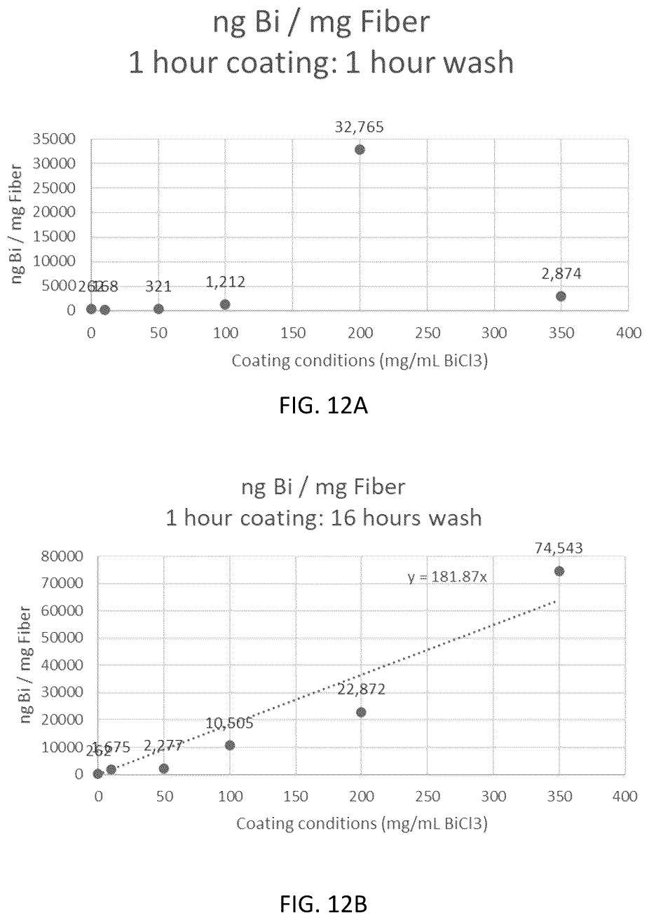

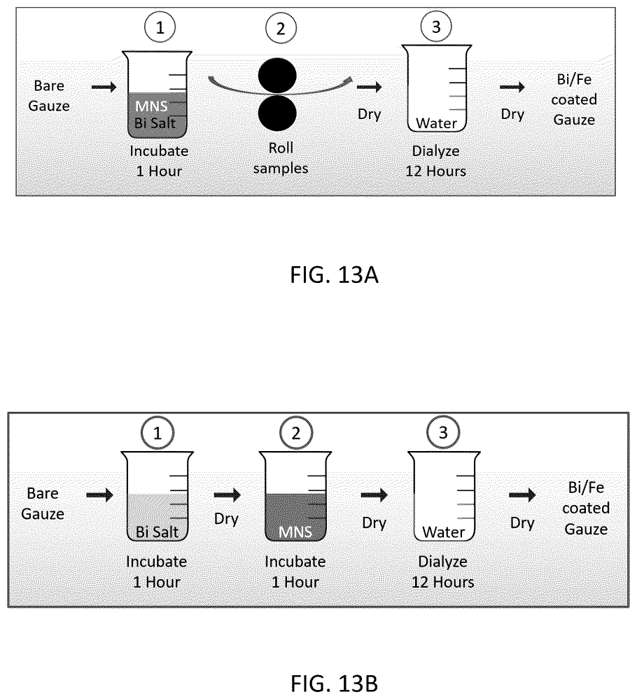

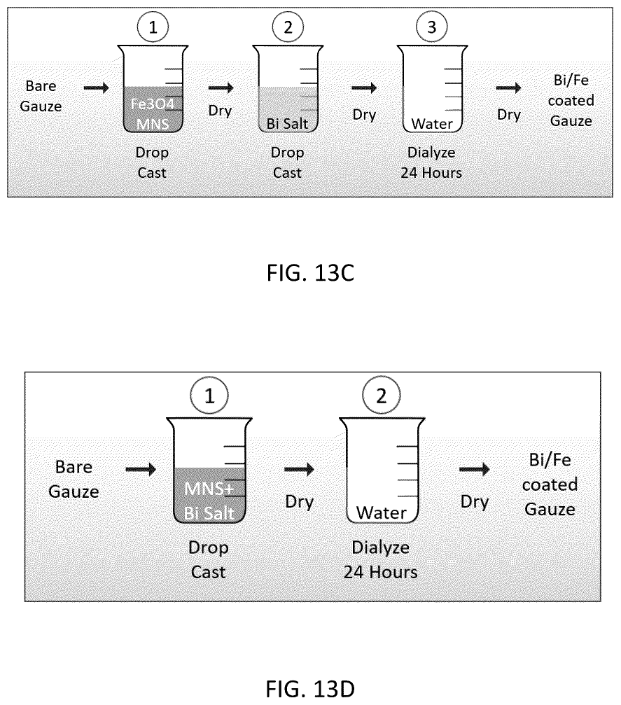

Various loading conditions for BiCl₃ were tested, including varying the amounts of BiCl₃ used to coat the fibers, the duration of incubation, the duration of the wash step, and varying the order in which fibers were coated with BiCl₃ and MNPs (e.g. coated simultaneously, or coated separately. Exemplary loading conditions for bismuth fibers are shown in FIG. 12. Exemplary methods tested to coat the fibers are shown in FIG. 13. Tested conditions for BiCl₃ loaded fibers resulted in visible MR contrast, but contrast was considered insufficient for reliable detection by CT. Accordingly, alternative methods for generating bismuth coated fibers were explored, as described below in Example 2.

### Example 2

How efficient a material attenuates x-rays for CT is dependent on the mass absorption coefficient μ which can be determined by Equation 1.1.

$$μ ∼ \frac{ρ {Z}^{4}}{A {E}^{3}} \qquad Eq . 1.1$$

In this equation, Z is the atomic number, p is density of the material, A is the atomic mass, and the energy of the X-rays used is given by E. Increasing atomic number drastically improves attenuation of the X-ray by the material in question. For this reason, large and dense elements at the bottom end of the periodic table offer the best X-ray attenuation, and bismuth, the largest non-radioactive element, with a density of 9.5 g cm⁻³ offers over ten times the attenuation of barium.

MR contrast agents use paramagnetic species, including gadolinium, manganese, and iron-oxide nanoparticles to alter the relaxation the protons from water and organic molecules in biological tissue when placed in an external magnetic field and perturbed by an RF pulse. Molecular agents containing manganese and gadolinium are used as T₁ contrast agents, and produce positive MR contrast, while iron-oxide nanoparticles are generally used as T₂ contrast agents and afford negative contrast. Gadolinium and manganese can have toxicity issues.

To prepare surgical gauze that can be detected by both MR and CT, a gauze was coated with both an MR active component and a CT active component. In this example, Fe₃O₄ and Bi NPs were used. Bismuth nanoparticles were used to increase the amount of bismuth that could be coated onto gauze samples, as imaging demonstrated that >12% bismuth by mass was necessary for visible X-ray CT contrast on gauze samples. Super-Paramagnetic Fe₃O₄ nanoparticles were chosen as the MR contrast media given their low cost, biological compatibility and low toxicity. Once coated, these particles were assessed by both T₂ weighted MR and X-ray CT to determine whether the investigated conditions afford sufficient contrast by both imaging modalities.

Preparation Bi/Fe₃O₄ NPs Coated Gauze

### Synthesis of Bi Nanoparticles (NPs):

Bismuth (Bi) NPs were synthesized via chemical reduction method. Bi(NO₃)₃ was used as Bi precursor, NaBH₄ was used as a reducing agent and polyvinylpyrrolidone (PVP) was used as a capping agent/surfactant. In a typical procedure, 300 mg of PVP and 100 mg of Bi(NO₃)₃ were dissolved in a mixture of 10 mL of glycerol and 5 mL of ethanol at 80 C. Then, 50 mg NaBH₄ was added quickly to the above mixture. The resulting mixture was stirred for another 10 mins. After the reaction, the products were collected by centrifugation and washed with ethanol three times, and then finally suspended in ethanol.

Synthesis of Fe₃O₄ Nanoparticles:

Fe₃O₄ NPs were synthesized via coprecipitation method. FeCl₃·6H₂O and FeCl₂·4H₂O were used as Fe precursor and NaOH was used as a precipitating agent. In a typical procedure, 54 mg of FeCl₃·6H₂O, 20 mg of FeCl₂·4H₂O were dissolved in a mixture of 10 mL of DI water at 80 C. Then, 32 mg of NaOH was added to the above mixture. The resulting mixture was stirred for another 30 mins. After the reaction, the products were collected by centrifugation and washed with DI water three times, and then finally dispersed in water.

Preparation of Bi/Fe₃O₄ NPs Coated Gauze

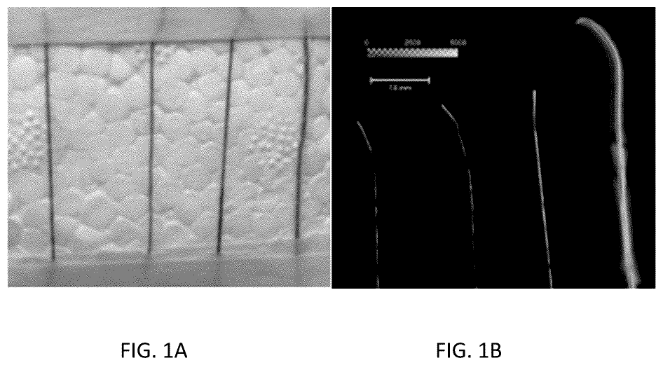

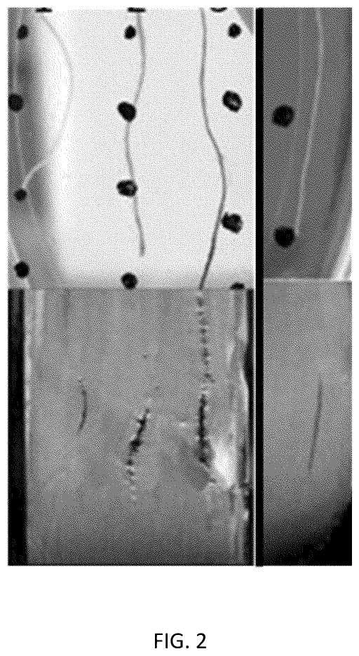

FIG. 1 shows various methods by which Bi/Fe₃O₄ NPs coated Gauze was prepared. FIG. 2 shows images comparing the resulting gauze prepared by the various procedures highlighted in FIG. 1. In a typical procedure, a clean gauze sample of size 1“xl” was dip coated in Bi NPs suspension for 1 min. Then it was passed through a wringer to remove excess solvent. The process was repeated 3 times and then the Bi NPs coated gauze was dried in air. Then, the gauze was coated with Fe₃O₄ NPs using the same procedure. Finally, the gauze sample was dialyzed in water for 24 hours and dried in air and used for further characterization.

### Results

Coating and Evaluation of Surgical Gauze with MR and CT Active Nanoparticles

Gauze samples were coated with Fe₃O₄ or bismuth nanoparticles and evaluated by T2 weighted MR imaging or X-ray CT respectively to determine the desired range of coating concentrations for further evaluation. Gauze threads (i.e. fibers) coated with BiNPs were secured to Styrofoam and imaged by X-ray CT and compared to the industry standard barium sulfate impregnated polymer strip. Note that the industry standard strip, also referred to as the commercially available strip, is substantially thicker than the single threads in the figures herein. Accordingly, the contrast seen in both CT and MR images for the single gauze threads (i.e. gauze fibers) should be viewed in the context of being substantially thinner/smaller than the industry standard strip. Results are shown in FIG. 1. ICP-MS analysis of these samples showed the loading of bismuth on the gauze was between 120 μg Bi per mg treated gauze and 180 μg per mg treated gauze for the samples in this study. SEM and EDX of threads after coating with BINPs and Fe₃O₄NPs reveals that Bi and Fe were coating each of the X-ray CT imaging of these samples shows loading ˜12% bismuth by mass is at the lower edge of detectable X-ray CT contrast, and the Bi loading increased beyond 18% Bi by mass leads to similar performance to the commercial standard. Gauze threads coated with Fe₃O₄ nanoparticles were suspended in agarose and imaged by T2 weighted MRI. Results are shown in FIG. 2. Samples coated with ˜2, 10, and 15 μg Fe per mg coated gauze (0.2%, 1% and 1.5% Fe by mass) demonstrated sufficient MR contrast for detection across the sampled range of coating concentrations.

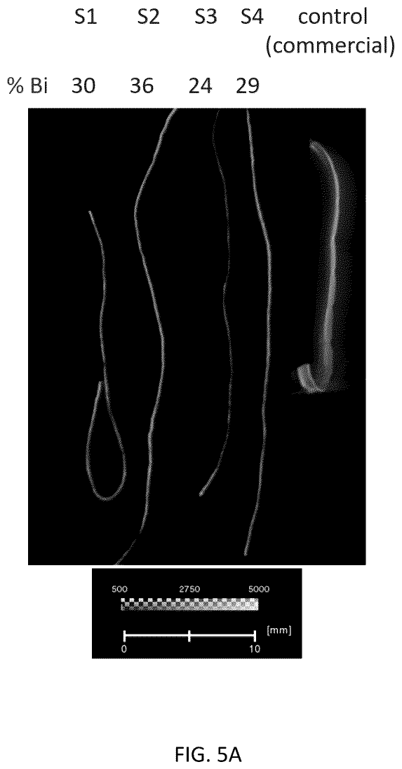

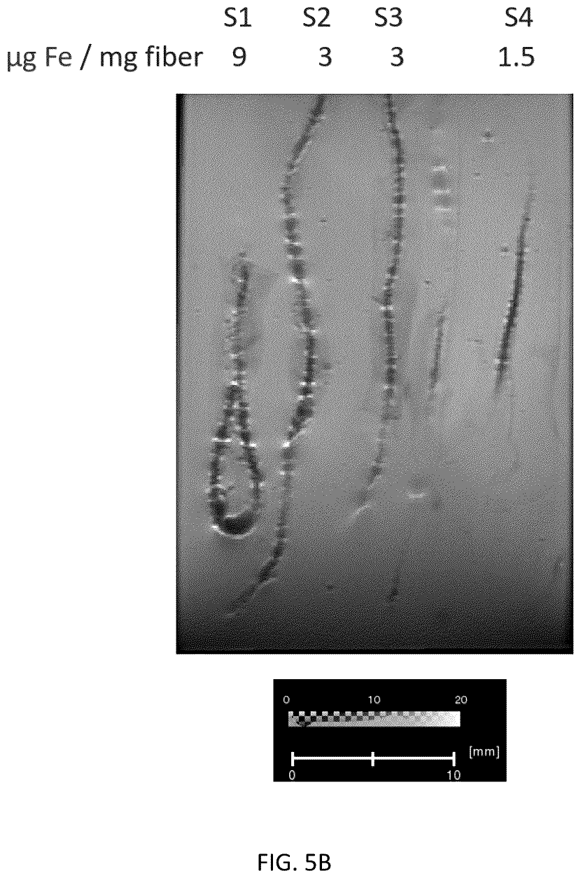

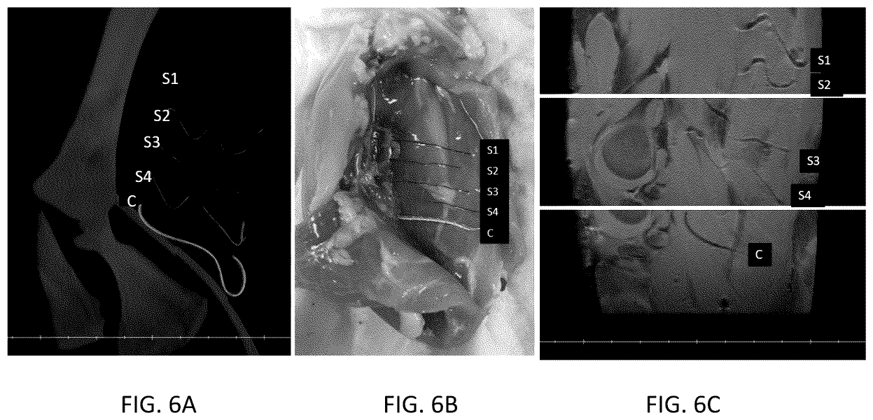

To determine whether the order of coating influences the efficiency of gauze coating with Bi and Fe, samples were coated with either BiNPs first or Fe₃O₄ nanoparticles first as shown in FIG. 3A. The gauze samples were treated with one cycle of Fe₃O₄ and five or ten cycles of BiNP coating. Samples from these four coating conditions were embedded in agarose and imaged by MR and X-ray CT. Specifically, a 2% agarose was prepared by dissolving 1 g of agarose in 50 mL boiling water. Agarose was poured and allowed to cool until it had set to a warm solid. Treated gauze fibers (approximately 3 cm in length) were selected from each gauze sample and laid on the surface of the agarose. To this, hot agarose was slowly added without disturbing the fiber samples and allowed to cool and set. CT and MRI imaging of fiber samples embedded in agarose gel as described here was expected to simulate results that would be obtained when imaging mammal and human subjects. These samples were submitted for MR and CT imaging. Data is shown in FIG. 5A (CT) and FIG. 5B (MR).

Bismuth and iron loading for these four coating conditions were 300, 360, 240, and 290 μg Bi and 9, 3, 3, and 1.5 μg Fe per mg coated gauze. MR and CT imaging showed that over 300 μg Bi and 3 μg Fe per mg treated gauze afforded contrast by MR and CT that was easily detectable in agarose. Contrast in CT was markedly increased compared to the CT contrast for bismuth salts, explored in Example 1.

### ICP-MS of Coated Gauze:

Treated gauze fibers (approximately 1.5 cm in length) were selected from each gauze sample. The exact mass of each fiber sample was recorded before they were digested in 300 μL concentrated nitric acid with 75 μL 30% hydrogen peroxide added. Samples were heated in a water bath at 65° C. for 1-2 days until the fiber was completely digested. Samples were diluted to 10 mL with ultrapure water and Fe and Bi concentrations were measured by ICP-MS against 100 ppb Fe and 100 ppb Bi standard solutions. These Bi and Fe concentrations were then multiplied by 10 mL to determine the total amount of Bi and Fe present in each fiber sample, and divided by the mass of each sample to determine the % of each sample that was composed of Bi and Fe.

Chicken and Steak with Embedded Gauze for MR and CT Sample Preparation:

To evaluate in-vivo efficacy of the coated gauze materials in various animal tissues, the three cuts of meat imaged were acquired from a local butcher. Chicken thigh, connective tissue bearing beef steak, and lean beef tenderloin with no connective tissue were the samples chosen for imaging.

Skin was removed from the chicken thigh, excess fat around the exterior of the connective tissue bearing steak was removed, and silverskin was removed from the tenderloin. A slit was cut into the meat, and treated gauze threads were laid into the slit along with commercially available BaSO₄ containing contrast strip. The slit in the meat was then closed after adding saline to prevent air bubbles around the samples. For the chicken thigh samples, coated gauze fibers were prepared as shown in FIG. 3A. For the beef steak and beef tenderloin samples, coated gauze fibers were prepared as shown in FIG. 3B. Namely, gauze was first treated with ten coats of BiNPs followed by 1, 3, or 5 coats of the iron oxide nanoparticles to improve iron loading and MR contrast.

FIGS. 5A-5C are exemplary images demonstrating in-vivo efficacy Bi/Fe₃O₄ NP coated gauze fibers placed in chicken thighs. FIG. 5A shows an exemplary CT image following placement of coated fibers within the tissue. FIG. 5B shows placement and alignment of the coated fibers within the tissue under natural lighting. FIG. 5C shows an exemplary MR image of the coated fibers following placement within the tissue. For both MR and CT imaging, coated gauze fibers can clearly be distinguished from the background provided by the chicken meat.

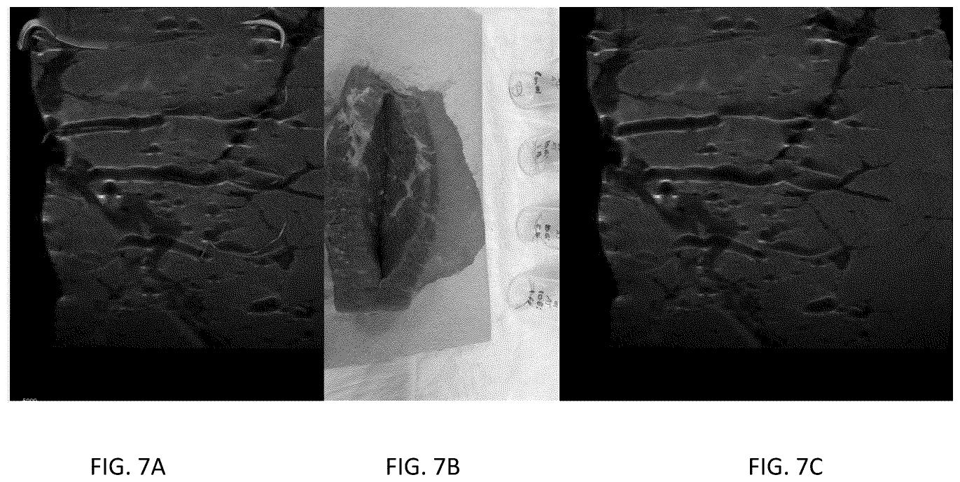

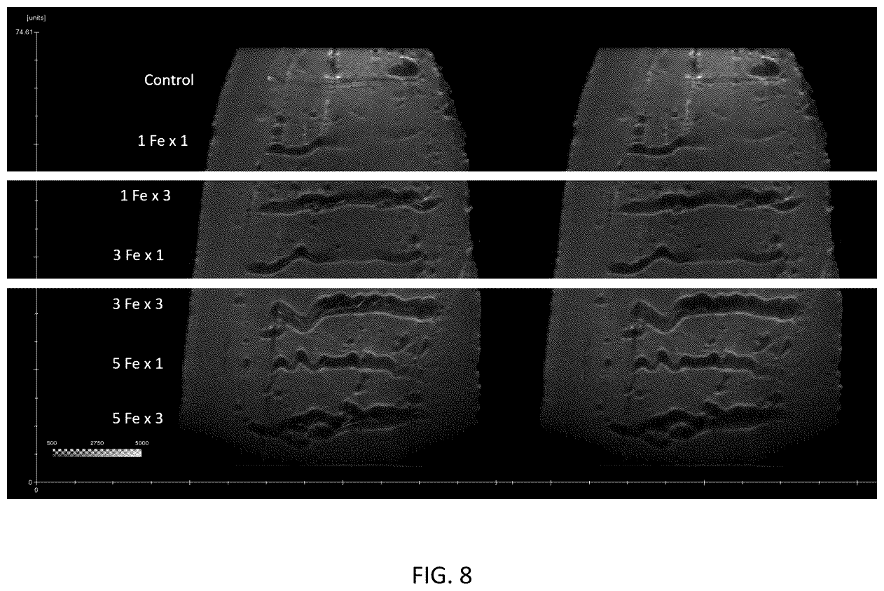

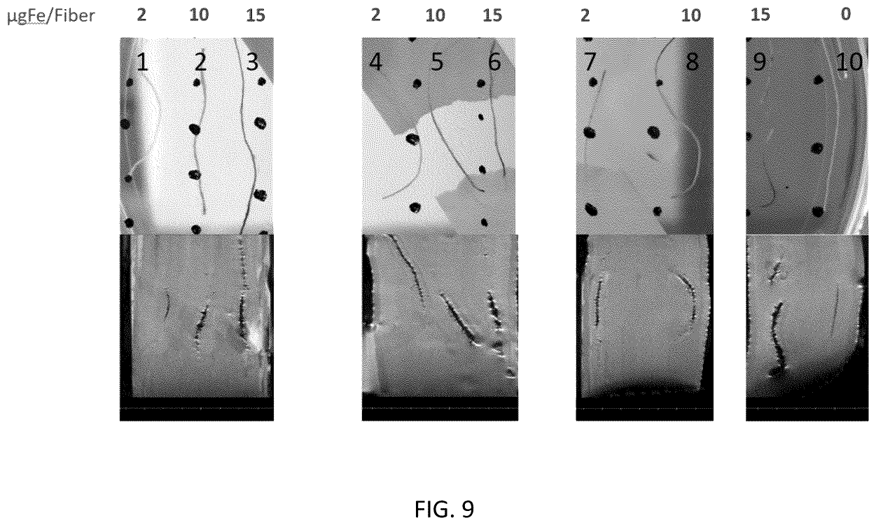

These samples were also imaged in two different type of steak, steak containing large amounts of connective tissue (FIG. 7) and steak containing very little connective tissue (FIG. 8). X-ray CT imaging revealed treated gauze samples were clearly visible in both meat conditions. MR imaging showed that treated gauze afforded sufficient T₂ contrast for detection. MR contrast enhancement was clearly visible in steak images with little connective tissue, and when several treated threads were twisted together the resultant MR contrast was extremely apparent.

In summary, a nanoparticle solution was used to create detectable CT and MR in cellulose gauze. Bismuth and iron oxide nanoparticles were bound to cellulose surgical gauze through hydrogen and coordinate covalent bonds. ICP-MS, T₂ weighted MR and X-Ray CT analysis demonstrated that between 200 and 300 μg Bi (e.g. 24-36% bismuth) and 2 and 15 μg Fe per mg coated gauze were sufficient to generate detectable contrast in model systems. Bismuth as a CT contrast agent allows for a thin coating of contrast agent on the fibers in gauze to provide effective CT detection. T₂ MR contrast from iron oxide MNPs can be visualized with adequate contrast against muscle. As described above, the results presented herein show efficacy for both CT and MR using a single thread, which is significantly smaller/thinner than the commercially available barium coated strip. Moreover, crumpling and twisting of treated threads together can enhance the contrast of treated gauze against other sources of T₂ contrast. Accordingly, an entire piece of gauze or other surgical material coated with the nanoparticles described herein would possess vivid contrast in vivo, even if a single thread became disrupted from the surgical material within the subject.

In some embodiments, the MR contrast agent can be a T₁ MR contrast agent. Gadolinium, manganese, and ultra small superparamagnetic iron oxide nanoparticles can all be used to generate T₁ positive contrast which may be easier to distinguish against different tissue types. Gadolinium and manganese leaching would need to be carefully managed to prevent their potential toxic side effects if that route is chosen.

Elements such as gold, tungsten, and osmium have densities from 19 to 23 g cm⁻³ compared to bismuth's 9.75 g cm⁻³ and may also be used as a CT contrast agent, which would allow up to a 50% greater CT contrast for equal coating concentrations. However, gold and osmium do come with a significant cost increase, and studies would be needed to determine maximum coating concentrations for tungsten nanoparticles.

Taken together, these results demonstrate that Bi/Fe₃O₄ NP coated gauze fibers (e.g. gauze fibers coated with bismuth nanoparticles and iron oxide nanoparticles) can be used in tissue from a variety of species with in-vivo efficacy for both CT contrast and MR contrast. Accordingly, surgical materials coated with bismuth nanoparticles and iron oxide nanoparticles, as described herein, can be used with high efficacy for multiple imaging modalities, including CT and MR. The surgical materials provided herein demonstrate significant advancements over existing materials, which are only detectable by CT and thus expose the subject to unnecessary radiation, and provide advancements for patient safety before and after surgery by using materials that can subsequently be detected by CT and/or MR to ensure that no unwanted materials remain within the subject after completion of surgery.

1. A surgical material comprising a detectable coating detectable by magnetic resonance imaging (MRI) and/or computerized tomography (CT), wherein the detectable coating comprises bismuth nanoparticles and/or iron oxide nanoparticles.

2. The surgical material of claim 1, wherein the detectable coating comprises Fe₃O₄ nanoparticles.

3. The surgical material of claim 1, wherein detectable coating comprises bismuth nanoparticles and iron oxide nanoparticles.

4. The surgical material of claim 3, wherein the detectable coating comprises bismuth nanoparticles and Fe₃O₄ nanoparticles.

5. The surgical material of claim 1, wherein the surgical material comprises a fibrous material.

6. Use of the surgical material of claim 1 in a subject.

7. A surgical material comprising a detectable coating detectable by magnetic resonance imaging (MRI) and computerized tomography (CT), wherein the detectable coating comprises bismuth nanoparticles and iron oxide nanoparticles.

8. The surgical material of claim 7, wherein the detectable coating comprises bismuth nanoparticles and Fe₃O₄ nanoparticles.

9. The surgical material of claim 7, wherein the detectable coating comprises at least 20% bismuth.

10. The surgical material of claim 9, wherein the detectable coating comprises 20% to 40% bismuth.

11. The surgical material of claim 7, wherein the detectable coating comprises about 1 μg iron to about 10 μg iron per milligram of the surgical material.

12. The surgical material of claim 7, wherein the surgical material comprises a fibrous material.

13. Use of the surgical material of claim 1 in a subject.

14. A composition comprising a plurality of bismuth nanoparticles.

15. The composition of claim 14, further comprising a plurality of iron oxide nanoparticles.

16. A method of making a surgical material comprising a detectable coating, the method comprising contacting the surgical material with the composition of claim 14.

17. A method of producing a bismuth nanoparticle, the method comprising dissolving a bismuth precursor and a surfactant in a composition comprising at least one alcohol, and reducing the bismuth precursor with a suitable reducing agent.

18. The method of claim 17, wherein the bismuth precursor comprises Bi(NO₃)₃ and the surfactant comprises polyvinylpyrrolidone.

19. The method of claim 17, wherein the composition comprises glycerol and ethanol.

20. The method of claim 17, wherein the reducing agent comprises NaBH₄.
